(19) United States
(12) Patent Application Publication
(10) Pub. No.: US 2020/0046217 A1
(43) Pub. Date: Feb. 13, 2020

(54) SYSTEMS, DEVICES, AND RELATED METHODS FOR RETRACTING TISSUE

(71) Applicant: Boston Scientific Scimed, Inc., Maple Grove, MN (US)

(72) Inventors: Art Butcher, Wellesley, MA (US); Paul Smith, Smithfield, RI (US); Ryan V. Wales, Northborough, MA (US); Jeff Gray, Sudbury, MA (US); Scott E. Brechbiel, Acton, MA (US); Sean P. Fleury, Minneapolis, MN (US); Laura E. Christakis, Framingham, MA (US); Gary A. Jordan, Litchfield, NH (US)

(21) Appl. No.: 16/533,059

(22) Filed: Aug. 6, 2019

Related U.S. Application Data
(60) Provisional application No. 62/715,521, filed on Aug. 7, 2018.

(51) Int. Cl. A61B 1/32; A61B 17/02; A61B 1/00

(52) U.S. Cl. CPC A61B 1/32; A61B 2017/00818; A61B 1/00135; A61B 17/0218

(57) ABSTRACT

A system may include a retractor movable between a radially-collapsed configuration and a radially-expanded configuration, a retraction tool having a tissue-engaging element and an anchor-engaging element, and a first member configured to form a releasable coupling with the anchor-engaging element at each of a plurality of spaced apart positions.

### CROSS REFERENCE TO RELATED APPLICATIONS

This application claims the benefit of priority under 35 U.S.C. § 119 to U.S. Provisional Patent Application Ser. No. 62/715,521, filed on Aug. 7, 2018, which is incorporated by reference in its entirety for all purposes.

### TECHNICAL FIELD

Various aspects of the present disclosure relate generally to tissue retraction. More specifically, the present disclosure relates to systems, devices, and related methods for retracting tissue.

### BACKGROUND

Technological developments have given users of medical systems, devices, and methods, the ability to conduct increasingly complex procedures on subjects. The removal of tissue in, for example, a subject's gastrointestinal tract, is a type of procedure in which difficulties may arise. One such area of difficulty involves removing a lesion from tissue. In order to remove the lesion, the user may retract tissue at or around the lesion. This retraction may enable the user to clearly observe a cutting plane for removing the lesion. Having this visualization may aid in preventing unwanted incision errors, such as severing vessels. The visualization also may aid in ensuring that as much of or all of the lesion is removed.

Endoscopic Submucosal Dissection (ESD) is a technique for removing polyps and tumors from the gastrointestinal tract. There are three distinct steps associated with an ESD technique, including injection at the target tissue site, cutting circumferentially around the target tissue, and dissecting beneath the submucosa of the lesion. ESD allows for the target tissue to be removed en bloc, improving the accuracy of the histopathologic assessment. Physicians are also able to resect deeper into the tissue, possibly removing cancers that have invaded deeper into the tissue layers. Since an electrocautery device is used to resect tissue (as opposed to a snare), physicians who conduct ESD may remove lesions of many sizes and shapes.

One limiting factor to ESD is in the complexity of the procedure. ESD may result in tissue wall perforation if sufficient elevation and separation of the tissue layers is not achieved. The risk to patients deters many physicians from using ESD. Some physicians are willing to reposition patients to leverage gravity as they conduct ESD. However, older and larger patients are difficult to reposition, and doing so could further compromise the procedure. One current technique involves utilizing a cap to help position an endoscope under a tissue flap, and to use a hybrid cutting device with injection capabilities. However, this may position the optics of the endoscope immediately adjacent the tissue, resulting in poor recognition (poor visualization) of the endoscope location relative to the rest of the lumen or larger tumors. Such techniques also may result in poor visualization of tissue tension achieved, and poor visualization of tissues impeding the dissection process.

### SUMMARY

Aspects of the disclosure relate to, among other things, systems, devices, and methods for retracting tissue. Each of the aspects disclosed herein may include one or more of the features described in connection with any of the other disclosed aspects.

A system may include a retractor movable between a radially-collapsed configuration and a radially-expanded configuration, a retraction tool having a tissue-engaging element and an anchor-engaging element, and a first member configured to form a releasable coupling with the anchor-engaging element at each of a plurality of spaced apart positions.

The system includes an overtube having a lumen configured to receive an endoscope, wherein the retractor is disposed at or adjacent to a distal end of the overtube. The first member is a rail that provides rigidity to the retractor when the retractor is in the radially-expanded configuration. The retractor includes the first member. The plurality of spaced apart positions are longitudinally spaced apart positions. The plurality of spaced apart positions include radially spaced apart positions. The retraction tool includes an elastic tether coupling the tissue-engaging element with the anchor-engaging element. The anchor-engaging element is a hook, and the first member includes a plurality of spaced apart rings each configured to receive the hook. One of more of the rings is biased radially outwardly from the first member. The anchor-engaging element includes hooks or loops, the first member includes a support having hooks or loops, and the anchor-engaging element and the support form a Velcro-type fastener. The first member is adapted to hold a sliding position of the anchor-engaging element. The sliding position of the anchor-engaging element is incremental and includes a plurality of teeth. The overtube includes one or more helical tracks having balls disposed therein, wherein movement of the balls through the one or more helical tracks drives the overtube longitudinally relative to the endoscope. The system further includes a handle configured to drive the balls through the one or more helical tracks, wherein rotation of the handle in a first direction causes the overtube to move distally relative to the endoscope, and rotation of the handle in a second direction opposite of the first direction causes the overtube to move proximally relative to the endoscope. The system further includes the endoscope, wherein the endoscope includes one or more helical tracks having a corresponding pitch as the one or more helical tracks of the overtube.

A system may include an overtube having a lumen configured to receive an endoscope, a retractor at or adjacent to a distal end of the overtube, the retractor being movable between a radially-collapsed configuration and a radially-expanded configuration, and a tool having a shaft and a tissue-engaging element coupled to a distal end of the shaft by a tether, wherein the distal end of the shaft is sloped relative to a proximal end of the shaft.

The tether is an elastic tether.

A method of treating a patient may include radially expanding a retractor while the retractor is adjacent to a target area in a body lumen of the patient, grasping tissue with a tissue-engaging element of a retraction tool, and tensioning the tissue by securing an anchor-engaging element, coupled to the tissue-engaging element, to a first anchor element of a plurality of spaced apart anchor elements.

The method further includes adjusting the tension of the tissue by securing the anchor-engaging element to a second anchor element of the plurality of spaced apart anchor elements. Before the radially expanding step, the method includes: positioning a distal end of an endoscope adjacent to the target area in the body lumen; and extending an overtube including the retractor over the endoscope so that the retractor is adjacent to the target area.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a perspective view of a system for operatively treating gastrointestinal disorders in a minimally-invasive manner, shown in a collapsed position.

FIG. 2 is a longitudinal cross-sectional view of the system of FIG. 1.

FIG. 3 is a perspective view of the system of FIG. 1, in an expanded position.

FIG. 4 is a perspective view of the system of FIG. 1 being inserted over the proximal end of an endoscope.

FIG. 5 illustrates insertion of the endoscope of FIG. 4 through a body lumen.

FIG. 6 is a perspective view showing the system of FIG. 1 being further advanced over the endoscope of FIG. 4.

FIG. 7 is a perspective view showing the system of FIG. 1 fully advanced over the endoscope of FIG. 4 to a desired position adjacent to target tissue.

FIGS. 8 to 14 show various other tissue retraction systems according to the disclosure.

### DETAILED DESCRIPTION

The present disclosure is drawn to systems, devices, and methods for retracting tissue. Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. The term “retraction” may refer, for example, to positioning tissue to expose and/or visualize a cutting area or plane for removing the tissue. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Embodiments of the present disclosure may be used for providing traction on target tissue in an endo-luminal space. In particular, some embodiments combine a luminal retraction (space-creating) apparatus with a tissue retraction device. The luminal retraction apparatus may include a retraction mechanism (e.g., a tissue clip), a scaffold structure (retractor) delivered by an endoscope, and at least one rail for translation of the retraction mechanism. The retractor may be delivered over the endoscope to the target tissue site. The overall system may include a mechanism for coupling a retraction mechanism, such as a tissue clip, to one or more anchored points on the rail. The retractor may be movable between open (expanded) and closed (retracted) states, and may include at least one opening (open access feature) that may be positioned adjacent to a target site. The retractor could be metallic, plastic, or include a shape memory metal, a shape memory polymer, a polymer, or any combination of materials. The retractor also may include a mechanism for anchoring to tissue at the desired site. This anchoring feature could take the form of barbs, hooks, spirals, outward radial force, and any other feature to secure the retractor to tissue.

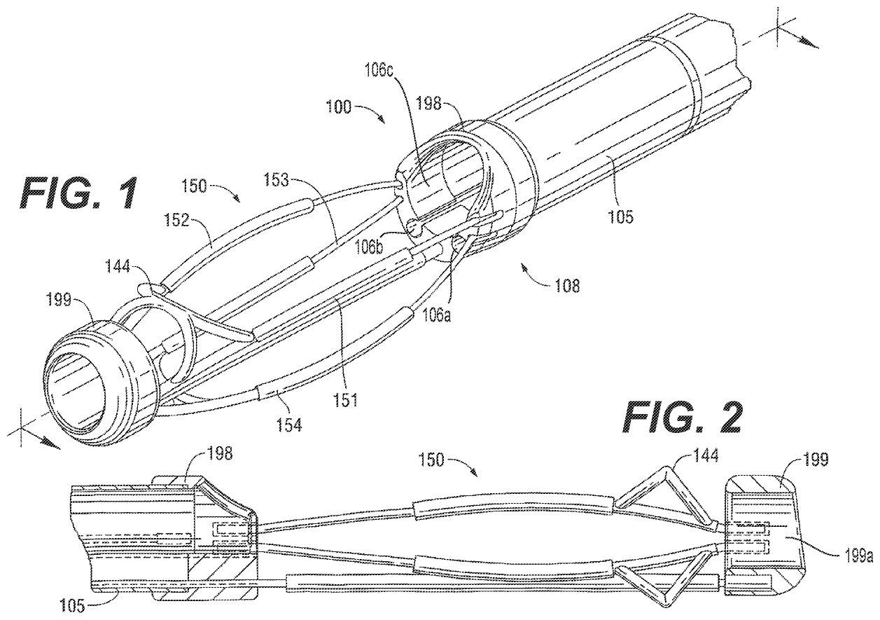

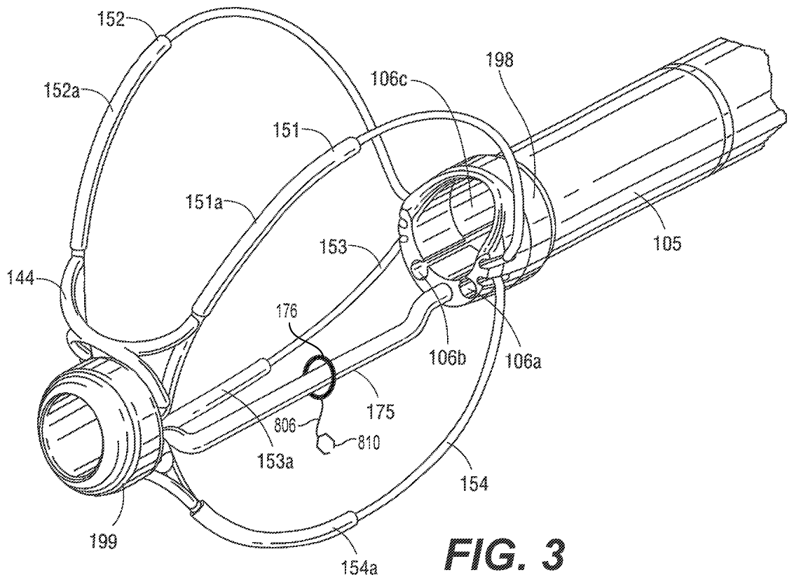

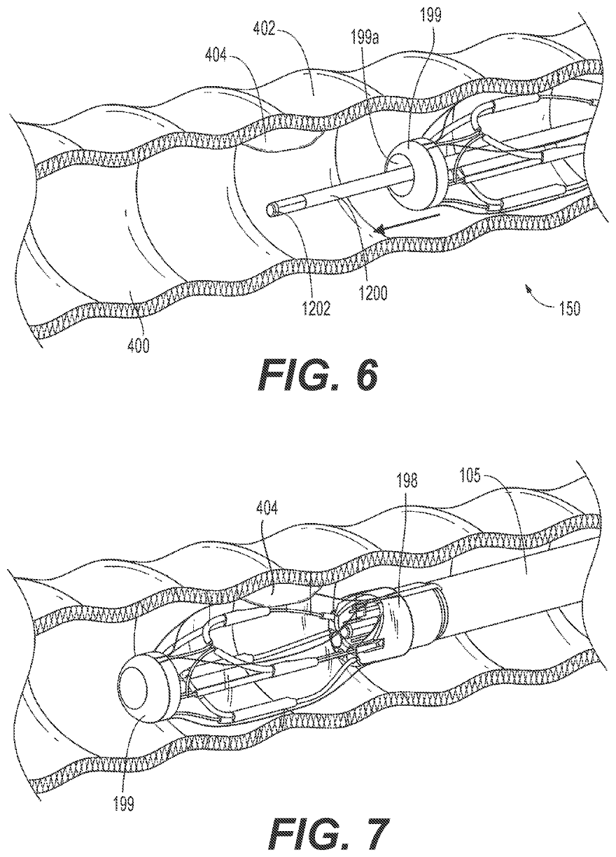

FIGS. 1-3 show a system 100 including a flexible overtube 105 for receiving one or more tools and an endoscope 1200 (shown in FIGS. 4-7). Overtube 105 may extend from a proximal end (not shown) toward a distal end 108, and may include tool lumens 106a, 106b, and a central lumen 106c for receiving endoscope 1200.

The tool inserted through tool lumens 106a and 106b may be any tool known to one of skill in the art. For example, the tool may include a grasper, a forceps, a snare, a scissor, a knife, a dissector, a clamp, an endoscopic stapler, a tissue loop, a clip applier, a suture-delivering instrument, or an energy-based tissue coagulator or cutter. Although two tool lumens 106a and 106b are illustrated, it should be appreciated that a system with more than two tool lumens or with only one tool lumen may be utilized. Additionally, endoscope 1200 itself may include one or more lumens for delivering working instruments and tools to a treatment site.

System 100 includes a reversibly-expandable retractor 150, as shown in FIG. 1, that expands to form a treatment space or working chamber in a patient. Retractor 150 may include retractor elements (legs) 151, 152, 153, 154, with a proximal coupler 198 and a distal coupler 199 each connected to retractor elements 151-154. The treatment space is formed to create a sufficient working area for performing a resection or dissection procedure, to enhance maneuvering and manipulation of the individual tools, and/or to enable tissue triangulation.

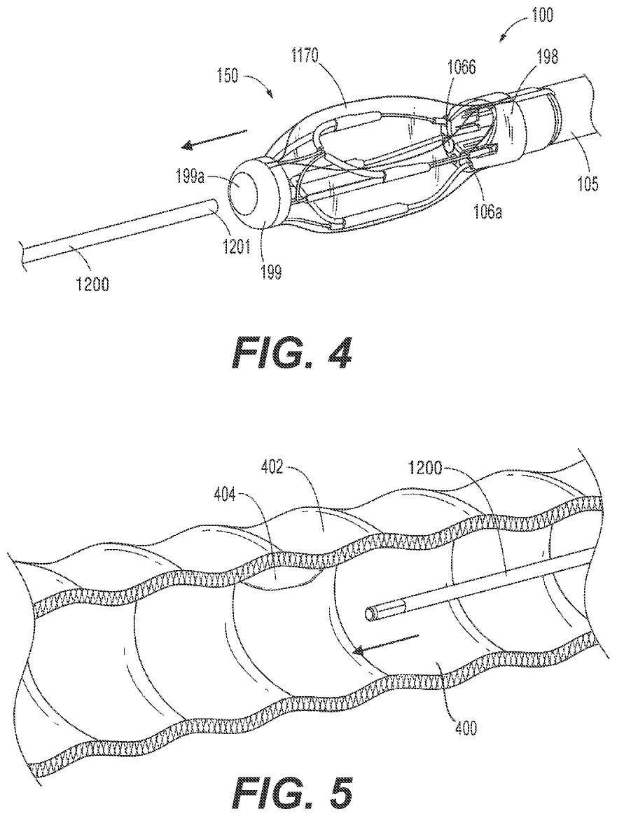

Distal coupler 199 is shown in the shape of a ring, although it may be virtually any shape desirable to one of skill in the art, such as a cone, a hemisphere, a sphere, and the like. Distal coupler 199 may include a port 199a for passage of endoscope 1200 beyond the distal end of system 100. Alternatively, distal coupler 199 may not include a port 199a, and distal coupler 199 may be closed. In some embodiments, proximal coupler 198 may be moved toward distal coupler 199, distal coupler 199 may be moved toward proximal coupler 198, or both couplers 198 and 199 may be moved toward each other, to reduce their distance apart and force retractor elements 151-154 radially outward from the collapsed position shown in FIG. 1 to the expanded position shown in FIG. 3. Retractor 150 may be repeatedly moved between its expanded and retracted positions as desired by adjusting the distance between couplers 198 and 199. Such controlled expansion of retractor elements 151-154 also may be achieved by operatively coupling the proximal ends of retractor elements 151-154 to an actuator that can be moved proximally and distally. Alternatively, retractor elements 151-154 may be composed of a material, e.g., shape memory material, to automatically expand when exposed from a catheter or sheath. In other words, retractor elements 151-154 may be biased into the expanded configuration shown in FIG. 3, and may be retained in the collapsed configuration shown in FIG. 1, when contained in a lumen of the catheter or sheath.

In some embodiments, retractor 150 may be reversibly stabilized by stiffening an otherwise flexible arrangement of retractor 150. The stabilization of retractor 150 may, in some embodiments, include a stabilizer having, for example, an at least substantially-rigid rail 175 to support the expanded retractor 150. Rail 175 may be substantially rectangular in cross-section, substantially circular in cross-section, or of other cross-sectional shapes. Rail 175 may include a same material or a stiffer material than retractor elements 151-154. Rail 175 may limit deflection of the distal end of system 100, which would otherwise occur by pressure exerted on the distal end by a body lumen wall. Rail 175 may be a straight component comprising a rigid material, for example stainless steel or another metal or alloy, that is slidable in and out of overtube 105. A ring or other sliding member 175a may slide along rail 175 (or any other rail described with respect to the embodiments and examples herein). Sliding member 175a may frictionally engage with rail 175 so that it has an infinite or near-infinite number of positions. Alternatively, sliding member 175a may slide incrementally and rest in various grooves or notches positioned along rail 175. A tether 806 and anchor 810 (described in further detail below) may be coupled to sliding member 175a. Alternatively, any other suitable tool, anchor, or engagement element described herein may be attached to sliding member 175a for anchoring and/or engaging tissue.

A bridge member 144 may be utilized to add stability to retractor 150. For example, retractor 150 may include bridge member 144 configured to maintain a desired orientation of the retractor elements during the expansion. As shown in FIGS. 1-3, bridge member 144 is attached to the two retractor elements (151, 152) for limiting side-to side movement of those retractor elements 151, 152. Bridge member 144 also may include a second bridge section connected to bridge member 144 and to retractor elements 153 and 154. Bridge member 144 may be a separate component or may be integrally formed with one or both of retractor elements 151, 152.

Retractor elements 151-154 each may have a covering (151a, 152a, 153a, 154a), which adds bulk to the respective retractor elements 151, 152, 153, 154 by increasing their effective cross-sectional diameter. Coverings 151a, 152a, 153a, 154a (FIG. 3) each extends over an intermediate portion of a respective retractor element 151-154, and may include a heat shrink tubing.

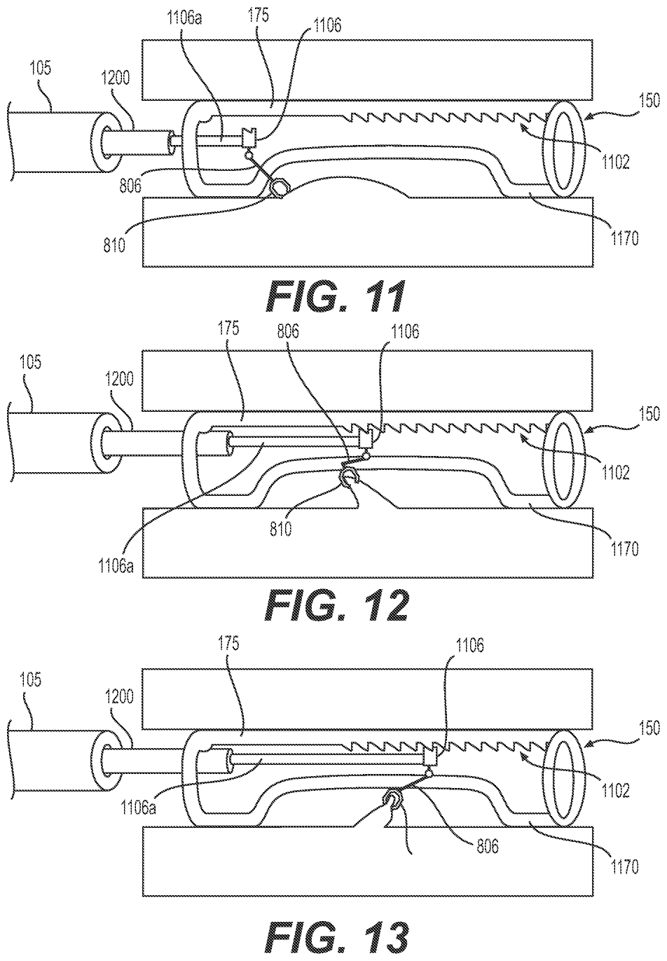

A covering or cover 1170 (shown in FIG. 4) may be provided at a distal end of overtube 105. Covering 1170 in the illustrated embodiment is mounted around the perimeter of proximal coupler 198 and distal coupler 199. In some embodiments, cover 1170 is pleated and sealed around couplers 198 and 199 by a heat shrink wrap. Cover 1170 is positioned around retractor elements 151-154 in the collapsed insertion position, with a side-facing opening in cover 1170 facing toward the target tissue, e.g., the lesion to be removed. That is, in the orientation of FIG. 7, the side-facing opening in cover 1170 faces upwardly toward a polyp 404. Cover 1170 may be configured to have an opening in the collapsed position, or, alternatively, it may be provided with a slit which may be opened due to stretching when retractor elements 151-154 are moved to the expanded position. In one embodiment, the edges of cover 1170 may be attached to retractor elements 151-154 so that cover 1170 moves with retractor elements 151-154. When the target tissue is removed by the endoscopic instruments described herein, the removed tissue may be placed within cover 1170, and cover 1170 may be closed, e.g., by a suture or string to encapsulate the tissue and prevent leakage and seeding during removal from the body lumen. Cover 1170 may provide a smooth and atraumatic surface for the delivery of retractor 150 to the target site. Cover 1170 also helps to prevent unwanted tissue, e.g., the luminal walls, from entering through the spaces between retractor elements during a surgical procedure.

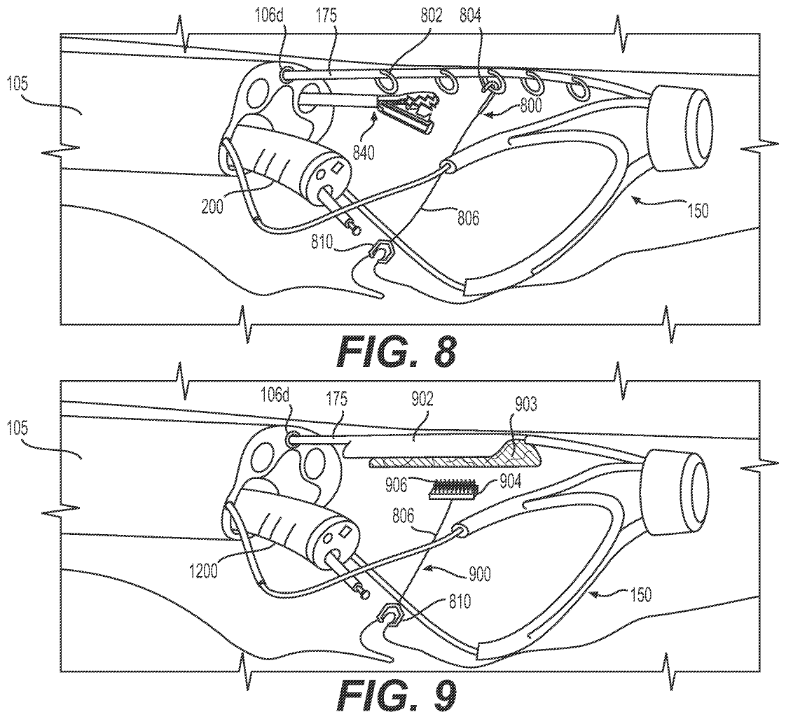

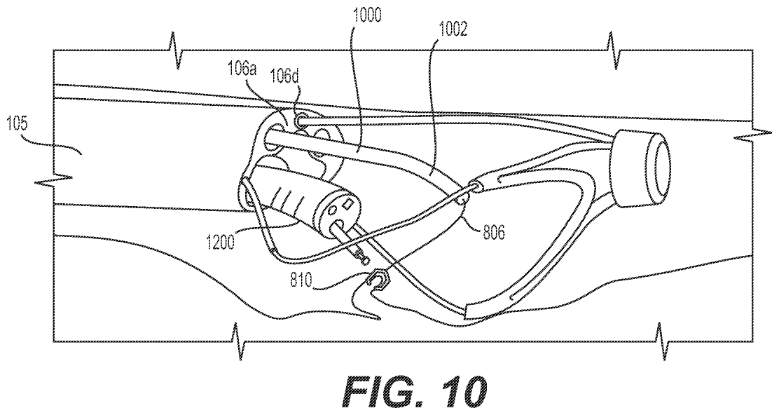

The use of system 100 will now be described with reference to removing a lesion, such as a polyp, from a body lumen wall. Turning first to FIGS. 4 and 5, overtube 105 can be loaded onto a proximal end 1201 of endoscope 1200, before endoscope 1200 is inserted through a lumen 400 in colon 402 to remove target polyp 404. Endoscope 1200 may be a distal viewing scope with a wide distal viewing area of about 150-170 degrees so polyp 404 and the surrounding area may be visualized. After placement of endoscope 1200 adjacent to the target issue (slightly proximal of target polyp 404), overtube 105 is further advanced over endoscope 1200 as shown in FIG. 6 until it reaches the target site as shown in FIG. 7, with retractor 150 aligned with polyp 404. When retractor 150 is at the position in the body shown in FIG. 7, it may be expanded to create the desired working space.

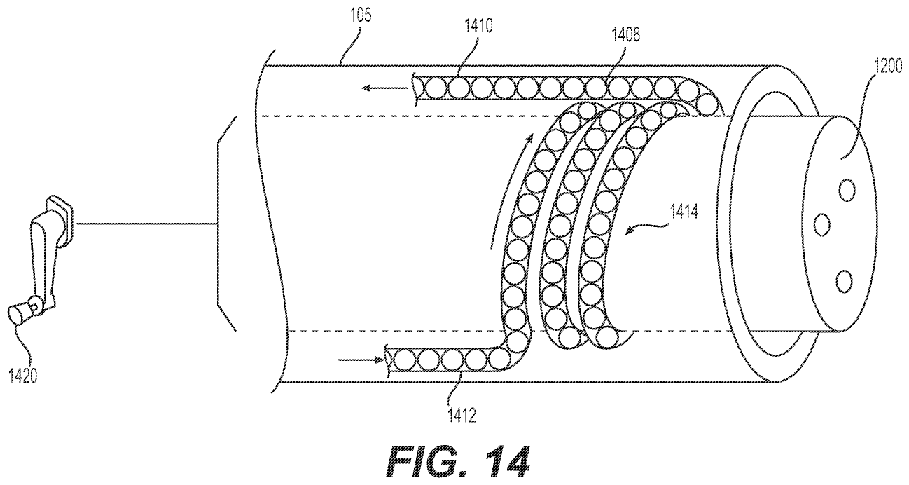

Once expanded, endoscope 1200 may be drawn proximally between couplers 198 and 199 to provide additional work space, and to provide a view of the work space (via the optics of endoscope 1200). Targeted polyp 404 (or other tissue) may be placed circumferentially between two circumferentially adjacent retractor elements. Then, tissue clips may be opened, closed, rotated, and coupled to target polyp 404, at which point cutting and injection devices may be used to perform, e.g., endoscopic submucosal dissection (ESD). As discussed further below, a tether may be used to selectively couple a tissue clip to rail 175 at different locations (e.g., longitudinal positions), allowing for users to change the angle of traction being generated on the grasped tissue. Thus, the following FIG. 8-13 show embodiments where rail 175 includes features to support and secure a traction device (e.g., a tissue clip) at various longitudinal positions within system 100 and retractor 150, to enable an operator to change the position, angle, and tension of grasped tissue. While each embodiment shown only depicts one rail, any embodiment shown could contain multiple circumferentially and/or radially spaced apart rails 175 to provide an operator with additional options of directional translation for tissue. Thus, a retraction device (e.g., a tissue clip) could be anchored to circumferentially, radially, and/or longitudinally spaced apart locations. Additionally, rail 175 could itself be adjustable by the user to a desired direction. Translation along rail 175 could include positional locking and/or incremental movement. Translation of the traction device (e.g., a tissue clip) could be accomplished by manipulation along the rail by a secondary instrument (e.g., jaws of a grasper 840 shown in FIG. 8).

FIG. 8 shows an embodiment of system 100, where rail 175 includes one or more anchors 802. Anchors 802 may be rings, loops, rungs, or eyelets, for example. In one embodiment, anchors 802 may be flexible to enable anchors 802 to collapse when rail 175 is disposed within a lumen 106d of overtube 105. In another embodiment, anchors 802 may include a shape memory material that is biased into a radially-expanded condition. Anchors 802 may be reciprocally movable between a first, collapsed configuration (not shown) and a second, expanded configuration (shown in FIG. 8). In the first configuration, the plurality of anchors 802 may be constrained within lumen 106d, collapsed against rail 175 due to the pressure exerted by the wall defining lumen 106d. Anchors 802 may be transitioned from the first configuration to the second configuration by moving rail 175 distally relative to overtube 105 and out of lumen 106d. Once rail 175 extends distally past distal end 108 of overtube 105, a constraining force acting on anchors 802 may be removed, and anchors 802 may expand radially outward. In one embodiment, anchors 802 may be partial rings having a circumferential opening that completely separates a first end of the partial ring from a second end of the partial ring.

Anchors 802 may be used in conjunction with a tissue clip 800. Tissue clip 800 may include an engagement element 804, an anchor 810 for embedding into or otherwise grasping tissue, and a tether 806 for linking engagement element 804 to anchor 810. Engagement element 804 may be a hook or other similar structure for engaging with anchors 802. That is, engagement element 804 and anchor 802 may have corresponding mating features. In one example, engagement element 804 and anchor 802 may be magnetically attracted to one another (via exposure of opposite poles). In another embodiment, engagement element 804 and anchor 802 each may include tacky surfaces. Anchor 810 may include, for example, a grasping element formed by jaws. It is contemplated that the jaws may move between an open position for receiving tissue and a closed position for grasping the received tissue. Any suitable positioning instrument (not shown) may be configured to actuate the jaws between the open position and the closed position. Anchor 810 also may include a coil with a sharp tip to embed into tissue, a barb, a spike, or another suitable tissue grasping element. Tether 806 may include a wire, a cord, a cable, an elastic band (e.g., a rubber band), a spring (e.g., a helical tension spring), a suture, a high carbon spring wire, a braided or wound filament, stainless steel, nitinol, spring steel, music wire, muscle wire, and/or any other suitable elongate member. Tether 806 may be metallic, polymeric, or a combination of metallic and polymeric.

An operator may use grasper 840 or another suitable tool to adjust the longitudinal, circumferential, and/or radial position of engagement element 804, and thus adjust the tension or orientation of the tissue grasped by anchor 810, by coupling engagement element 804 to different anchors 802.

FIG. 9 depicts a system similar to the system shown in FIG. 8, except that in the embodiment of FIG. 9, rail 175 includes a support 902 having a plurality of loops 903 (e.g., Velcro-type loops). In this embodiment, a tissue clip 900 may be substantially similar to tissue clip 800 described above, except that engagement member 804 may be replaced by an engagement member 904 having a plurality of hooks (Velcro-type hooks) 906. Thus, support 902 and engagement member 904 may form a hook-and-loop fastener, a hook-and-pile fastener, or a touch fastener. Support 902 and engagement member 904 each may include fabric strips having loops and hooks respectively, that, when pressed together, are removably fastened to one another support 902 may have a substantially greater length and surface area than engagement member 904. For example, Support 902 may be from 2 to 50 times longer than engagement member 904, although other suitable ratios also are contemplated.

An operator may use grasper 840 (shown in FIG. 8) or another suitable tool to adjust the longitudinal, circumferential, and/or radial position of engagement member 904, and thus adjust the tension or orientation of the tissue grasped by anchor 810, by coupling engagement member 904 to different positions on support 902.

In the embodiment shown in FIG. 10, an additional tool 1000 may be extended through overtube 105 via lumen 106a. In this configuration, tool 1000 extends from lumen 106a into the treatment space created by retractor 150. A tether 806 may extend from distal end 1002 of tool 1000. An opposing end of tether 806 may be coupled to an anchor 810 described above. Thus, tool 1000 itself may be used to provide additional tension or directional control as required. This could be achieved by extending, rotating, and/or articulating a shaft of tool 1000. Distal end 1002 of tool 1000 also may contain a preset bend so that tool 1000 may be articulated or rotated in another direction as it passes out of lumen 106a. This angle may be controlled based on how far an operator extends distal end 1002 from lumen 106a. It also is contemplated that tool 1000 may be extended through a lumen of endoscope 1200, instead of through overtube 105.

FIGS. 11-13 show yet another alternative embodiment, where rail 175 includes a plurality of teeth 1102 that engage with a pawl 1106. Retractor 150 is shown in simplified form in order to more clearly show the interaction between teeth 1102 and pawl 1106. Pawl 1106 is coupled to an anchor 810 by a tether 806. Once anchor 810 is secured to tissue, the tension of that tissue may be adjusted by sliding pawl 1106 distally along rail 175 via teeth 1102. Pawl 1106 may be manipulated and/or controller by a tool 1106a (e.g., a grasper, rod, or any other suitable tool). In one embodiment, it is contemplated that pawl 1106 travels along rail 175 only in one direction (e.g., only distally), as the interaction between pawl 1106 and teeth 1102 substantially prevents proximal movement of pawl 1106. In some embodiments, pawl 1106 may be released from teeth 1102 by actuation of a release mechanism, to allow pawl 1106 to be removed from the body. In another embodiment, pawl 1106 must travel an entire length of rail 175 to be released. Pawl 1106 may be coupled to a tool extended through either overtube 105 or endoscope 1200. Pawl 1106 may be magnetically attracted to teeth 1102 in some embodiments. In yet other embodiments, pawl 1106 may part of a head assembly containing a lumen that slides over teeth 1102 (e.g., a zip-tie configuration). In such an embodiment, pawl 1106 may be contained within the lumen.

Overtube 105 and endoscope 1200 are shown in FIG. 14 with retractor 150 removed for clarification. As shown in FIG. 14, overtube 105 may include a ball screw mechanism for providing enhanced control of the longitudinal motion between overtube 105 and endoscope 1200. The ball screw mechanism may grip the outer surface of endoscope 1200 to convey overtube 105 distally (and proximally) with precision and force. The ball screw mechanism may be operated by a user, e.g., physician, by actuating a rotary handle 1420, which drives balls 1408 through a circuit. The rotary motion of handle 1420 is converted to linear motion of overtube 105 via a spiral path of balls 1408 through tracks 1414 disposed on an inner circumferential surface of overtube 105. The rotation of handle 1420 in a first direction may move overtube 105 distally by moving balls 1408 through tracks 1414 in a first path (such as shown by the arrows in FIG. 14), while the rotation of handle 1420 in a second direction may move overtube 105 proximally by moving balls 1408 through tracks 1414 in a second path opposite of the first path. In some embodiments, the outer surface of endoscope 1200 may include one or more helical tracks having a same or similar pitch as tracks 1414 of overtube 105. In an alternative embodiment, handle 1420 could be replaced by a motor. Balls 1408 may be circulated through a closed circuit via conduits 1410 and 1412 connected to opposing ends of track 1414.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A system, comprising:
a retractor movable between a radially-collapsed configuration and a radially-expanded configuration;
a retraction tool having a tissue-engaging element and an anchor-engaging element; and
a first member configured to form a releasable coupling with the anchor-engaging element at each of a plurality of spaced apart positions.

2. The system of claim 1, further including an overtube having a lumen configured to receive an endoscope, wherein the retractor is disposed at or adjacent to a distal end of the overtube.

3. The system of claim 1, wherein the first member is a rail that provides rigidity to the retractor when the retractor is in the radially-expanded configuration.

4. The system of claim 1, wherein the retractor includes the first member.

5. The system of claim 1, wherein the plurality of spaced apart positions are longitudinally spaced apart positions.

6. The system of claim 1, wherein the plurality of spaced apart positions include radially spaced apart positions.

7. The system of claim 1, wherein the retraction tool includes an elastic tether coupling the tissue-engaging element with the anchor-engaging element.

8. The system of claim 1, wherein the anchor-engaging element is a hook, and the first member includes a plurality of spaced apart rings each configured to receive the hook.

9. The system of claim 8, wherein one of more of the rings is biased radially outwardly from the first member.

10. The system of claim 1, wherein the anchor-engaging element includes hooks or loops, the first member includes a support having hooks or loops, and the anchor-engaging element and the support form a Velcro-type fastener.

11. The system of claim 1, wherein the first member is adapted to hold a sliding position of the anchor-engaging element.

12. The system of claim 11, wherein the sliding position of the anchor-engaging element is incremental and includes a plurality of teeth.

13. The system of claim 12, wherein the overtube includes one or more helical tracks having balls disposed therein, wherein movement of the balls through the one or more helical tracks drives the overtube longitudinally relative to the endoscope.

14. The system of claim 13, further including a handle configured to drive the balls through the one or more helical tracks, wherein rotation of the handle in a first direction causes the overtube to move distally relative to the endoscope, and rotation of the handle in a second direction opposite of the first direction causes the overtube to move proximally relative to the endoscope.

15. The system of claim 13, further including the endo scope, wherein the endoscope includes one or more helical tracks having a corresponding pitch as the one or more helical tracks of the overtube.

16. A system, comprising:
an overtube having a lumen configured to receive an endoscope;
a retractor at or adjacent to a distal end of the overtube, the retractor being movable between a radially-collapsed configuration and a radially-expanded configuration; and
a tool having a shaft and a tissue-engaging element coupled to a distal end of the shaft by a tether, wherein the distal end of the shaft is sloped relative to a proximal end of the shaft.

17. The system of claim 16, wherein the tether is an elastic tether.

18. A method of treating a patient, the method comprising:
radially expanding a retractor while the retractor is adjacent to a target area in a body lumen of the patient;
grasping tissue with a tissue-engaging element of a retraction tool; and
tensioning the tissue by securing an anchor-engaging element, coupled to the tissue-engaging element, to a first anchor element of a plurality of spaced apart anchor elements.

19. The method of claim 18, further including adjusting the tension of the tissue by securing the anchor-engaging element to a second anchor element of the plurality of spaced apart anchor elements.

20. The method of claim 18, wherein, before the radially expanding step, the method includes:
positioning a distal end of an endoscope adjacent to the target area in the body lumen; and
extending an overtube including the retractor over the endoscope so that the retractor is adjacent to the target area.
